(19) United States
(12) Patent Application Publication
(10) Pub. No.: US 2025/0229090 A1
(43) Pub. Date: Jul. 17, 2025

(54) IMPLANTABLE MEDICAL DEVICE WITH A SLOT ANTENNA

(71) Applicant: NeuroPace, Inc., Mountain View, CA (US)

(72) Inventors: Alister Hoss, Sherman Oaks, CA (US); Natalya A. Bruner, Santa Cruz, CA (US); Stephen T. Archer, Sunnyvale, CA (US); Tygo A. Ebenhahn, San Francisco, CA (US)

(21) Appl. No.: 18/411,443

(22) Filed: Jan. 12, 2024

(51) Int. Cl. A61N 1/372; A61N 1/375

(52) U.S. Cl. CPC A61N 1/37217; A61N 1/375

(57) ABSTRACT

An implantable medical device (IMD) includes an enclosure formed of an electrically conductive material. The enclosure includes an outer wall and a slot that extends through the outer wall. The slotted enclosure forms a slot antenna. A dielectric element hermetically seals the slot. Communication circuitry within the enclosure is electrically coupled with the slot antenna.

### TECHNICAL FIELD

The present disclosure relates generally to antennas for implantable medical devices, and more particularly, to a slot antenna formed by an enclosure or housing of an implantable medical device.

### BACKGROUND

Active implantable medical devices are configured to communicate with external components wirelessly, such as via a form of telemetry. The communication may be desirable to, for example, download information acquired by and stored in the implanted medical device to an external component, such as patient-controlled external component. Alternatively, or additionally, an external component configured as a programmer may be brought into communication with the implantable medical device to obtain data from the device or to send data to the device from the programmer, such as new programming instructions that control whatever it may be that the implantable medical device is configured to do. For example, the implantable medical device may be programmed to measure electrographic signals sensed from the patient and/or detect electrographic events whenever such events occur in the electrographic signals or deliver a form of electrical stimulation to the patient.

A communication link between the implantable medical device and an external apparatus may be established with an intermediate device, such as wand for near-field or short-range telemetry. For example, the implantable medical device may have a telemetry coil that enables transmission and reception of signals, to or from an external apparatus, via inductive coupling. Alternatively, a communication link between the implantable medical device and an external component may be established with an antenna for a radio frequency (RF) link. Far field or long-range telemetry may obviate the need for the intermediate device (e.g., the wand) and allow the external apparatus to be further away from the implantable medical device than is the case with near-field telemetry.

### SUMMARY

An implantable medical device configured for implant in a body includes an enclosure formed of an electrically conductive material. The enclosure includes an outer wall having a slot that extends through the outer wall. The enclosure with the slot defines a slot antenna. A dielectric element hermetically seals the slot. Communication circuitry within the enclosure is electrically coupled with the slot antenna.

It is understood that other aspects of implantable medical devices will become readily apparent to those skilled in the art from the following detailed description, wherein various aspects of implantable medical devices are shown and described by way of illustration. As will be realized, these aspects may be implemented in other and different forms and its several details are capable of modification in various other respects. Accordingly, the following drawings and detailed description are to be regarded as illustrative in nature and not as restrictive.

### BRIEF DESCRIPTION OF THE DRAWINGS

Various aspects of implantable medical devices will now be presented in the detailed description by way of example, and not by way of limitation, with reference to the accompanying drawings, wherein:

FIG. 1 is an illustration of a neurostimulation system implanted in a patient, including a pair of leads and an implantable medical device (IMD) in the form of a neurostimulator with a slot antenna.

FIG. 2A is an illustration of a neurostimulator with a slot antenna formed by a metallic enclosure having a slot and a dielectric element that seals the slot.

FIG. 2B is a side illustration of the neurostimulator of FIG. 2A, with a portion of the metallic enclosure removed to show the interior of the enclosure.

FIG. 2C is an enlarged illustration of the cross-section along line 2C-2C of FIG. 2A.

FIG. 2D is a version of the illustration of FIG. 2C that shows the dielectric element separated from the metallic enclosure.

FIG. 3A is an illustration of the dielectric element of FIG. 2A.

FIG. 3B is a top view of the dielectric element of FIG. 3A.

FIG. 3C is a cross-section of the dielectric element along line 3C-3C of FIG. 3A.

FIG. 3D is an exploded view of the cross-section of the dielectric element of FIG. 3C.

FIG. 4A is a schematic cross-section of components of a slot antenna coupled to circuitry of the neurostimulator through a transmission line.

FIGS. 4B and 4C are detailed illustrations of connections between the transmission line and the dielectric element.

FIG. 5A is an illustration of a transmission line.

FIGS. 5B-5D are cross-section illustrations of the transmission line along lines 5B-5B, 5C-5C, and 5D-5D of FIG. 5A.

FIG. 6A is a schematic illustration of a slot antenna formed by a metallic enclosure having a slot.

FIG. 6B is a cross-section illustration along line 6B-6B of FIG. 6A.

FIGS. 6C-6D are illustrations of the slotted portion of the metallic enclosure of FIG. 6B with different configurations of a dielectric element that seals the slot.

FIG. 7 is a graph of radiation gain as a function of direction of radiation for a slot antenna of a neurostimulator of FIG. 2A.

FIG. 8 is a graph of peak gain as a function of radio frequency for a slot antenna of a neurostimulator of FIG. 2A.

FIG. 9 is an example graph of initial return loss as a function of radio frequency for a slot antenna of a neurostimulator of FIG. 2A.

### DETAILED DESCRIPTION

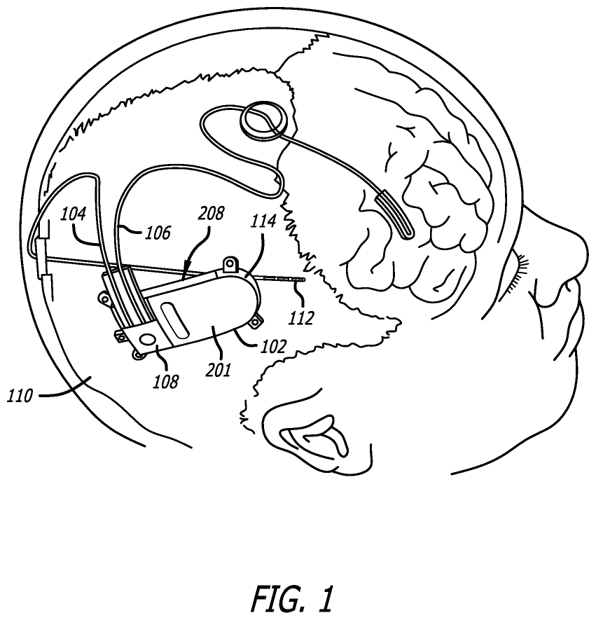

With reference to FIG. 1, an example implantable medical system in the form of a neurostimulation system is implanted in a patient. The neurostimulation system includes an implantable medical device (IMD), referred to going forward as a neurostimulator 102, and two electrode-bearing brain leads 104, 106. The neurostimulation system is configured to sense and record electrical brain activity, to detect electrographic events in the electrical brain activity, and to deliver responsive neurostimulation therapy. Responsive neurostimulation systems are described in, for example, U.S. Pat. No. 6,016,449 to Fischell, et al. for “System for Treatment of Neurological Disorders”, issued Jan. 18, 2000, U.S. Pat. No. 6,810,285 to Pless et al. for “Seizure Sensing and Detection Using an Implantable Device,” issued Oct. 24, 2004, and U.S. Pat. No. 6,690,974 to Archer et al. for “Stimulation Signal Generator for an Implantable Device” issued Feb. 10, 2004. Each of the '449, '285 and '974 patents is hereby incorporated by reference in their entirety.

The neurostimulator 102 is configured to be secured in an opening formed through the cranium 110. To this end, a tray or ferrule 114 is placed in the opening and secured to the cranium, and the neurostimulator 102 is placed in the ferrule. The neurostimulator 102 is oriented in the ferrule 114 such that the slotted side 201 of a slot antenna 208 faces outward from the interior of the cranium in a direction facing the exterior of the body, and is adjacent to the patient's scalp tissue.

With continued reference to FIG. 1, the neurostimulator 102 includes a lead connector 108 adapted to receive one or more of the brain leads, such as a deep brain or depth lead 104 and a cortical strip lead 106. The depth lead 104 is implanted so that a distal end of it is situated within the patient's neural tissue, whereas the cortical strip lead 106 is implanted under the dura mater so that a distal end of it rests on a surface of the brain. The lead connector 108 acts to physically secure the brain leads 104, 106 to the neurostimulator 102, and facilitates electrical connection to conductors in the brain leads coupling one or more electrodes at or near a distal end of the lead to circuitry within the neurostimulator 102.

The proximal portion of the deep brain lead 104 is generally situated on the outer surface of the cranium 110 (and under the patient's scalp), while the distal portion of the lead enters the cranium and is coupled to at least one depth electrode 112 implanted in a desired location in the patient's brain. The proximal portion of the cortical lead 106 is generally situated on the outer surface of the cranium 110 (and under the patient's scalp), while the distal portion of the lead enters the cranium. The distal portion of the cortical lead 106 includes at least one cortical electrode (not visible) implanted at a desired location on the patient's brain.

Long-range (wireless) telemetry is a form of communication between implantable medical devices (IMD) and external programmers and monitors. This communication can take place over several meters or even across rooms. Previous generations of systems used near-field inductive telemetry to communicate from implanted device to external programmer. In modern and emerging systems, radio frequencies are used for long-range telemetry allowing much longer distances of communication. Along with radio frequencies comes the demand for better, high-tuned antennas to support long-range telemetry.

An international standard for implant communication is the Medical Implant Communication Service) MICS, which operates at 401-406 MHz [ETSI, 2002]. Due to the frequency of this signal and the limited power restrictions imposed by the standard, conventional antenna designs for IMDs are placed outside the metallic housing within rigid encapsulation, such as epoxy, to keep the antenna structure rigid and immobile. New antenna designs that include the metallic housing of the IMD, and do not require encapsulation are disclosed below.

Neurostimulator with Slot Antenna

Disclosed herein is an IMD, e.g., a neurostimulator, with a slot antenna that is formed by an enclosure of the neurostimulator having a slot that extends through a wall of the enclosure. The slot antenna is configured to provide efficient radiation to operate in one or more frequency bands in a particular operating environment, wherein the slot antenna is adjacent to biological tissue. In some embodiments, the slot antenna operates in a frequency band in a range of at least one of the 2.4 GHz spectrum band (2400 to 2483.5 MHz) (e.g., Bluetooth) and the 5.8 GHz spectrum band (5.15 GHz to 5.85 GHZ) (e.g., WiFi). In some embodiments, the slot antenna operates in two frequency bands, including a first frequency band in a range of 2.4 GHz to 2.5 GHZ, and a second frequency band in a range of 5.15 GHz to 5.85 GHz. In some embodiments, the slot antenna has a peak radiation gain in a range of −8 dBi to −13 dBi when implanted under skin and operating in a frequency band of 2.4 GHz to 2.5 GHz. In some embodiments, the slot antenna 208 has a peak radiation gain in a range of −5 dBi to −15 dBi when implanted under skin and operating in a frequency band of 5.15 GHz to 5.85 GHz.

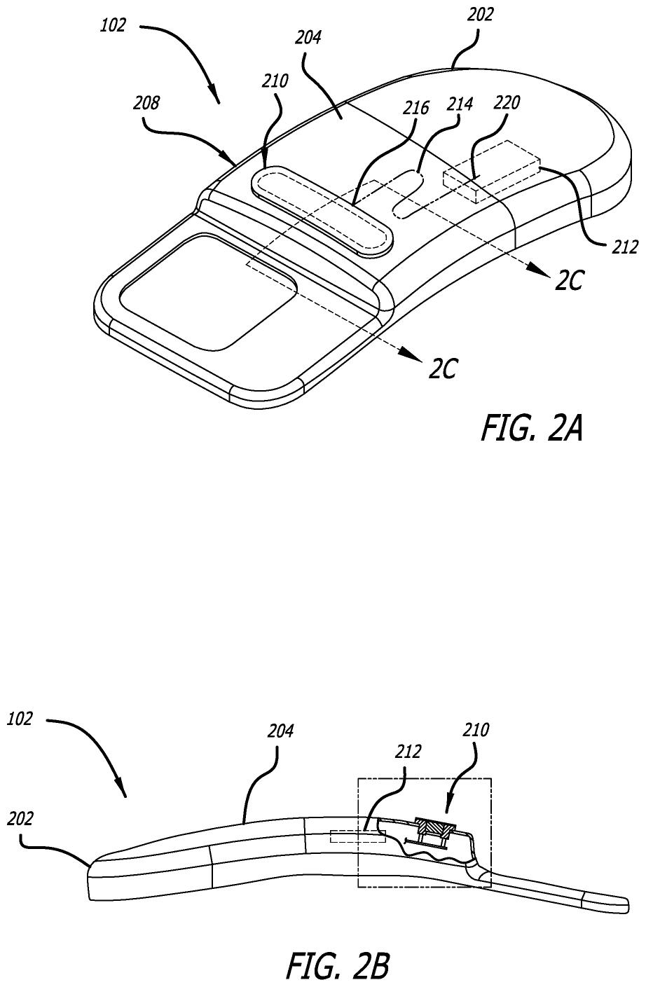

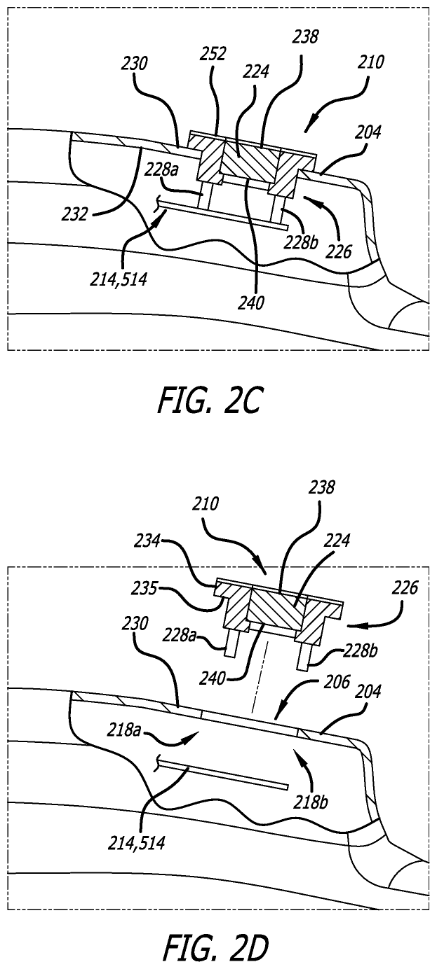

With reference to FIGS. 2A-2D, a neurostimulator 102 configured in accordance with embodiments disclosed herein includes a conductive metallic enclosure 202 having an outer wall 204 and a slot 206 that extends through the outer wall. The enclosure 202 can be formed of any biocompatible conductive metal, e.g., titanium, or any biocompatible conductive alloy, e.g., an alloy that includes titanium or an alloy that includes stainless steel. Note that in FIGS. 2A-2D some components of the neurostimulator 102 are not shown for clarity in illustrating other components. For example, a connector feedthrough is not included in the portion of the neurostimulator 102 where the lead connector 108 couples to the neurostimulator (as shown in FIG. 1).

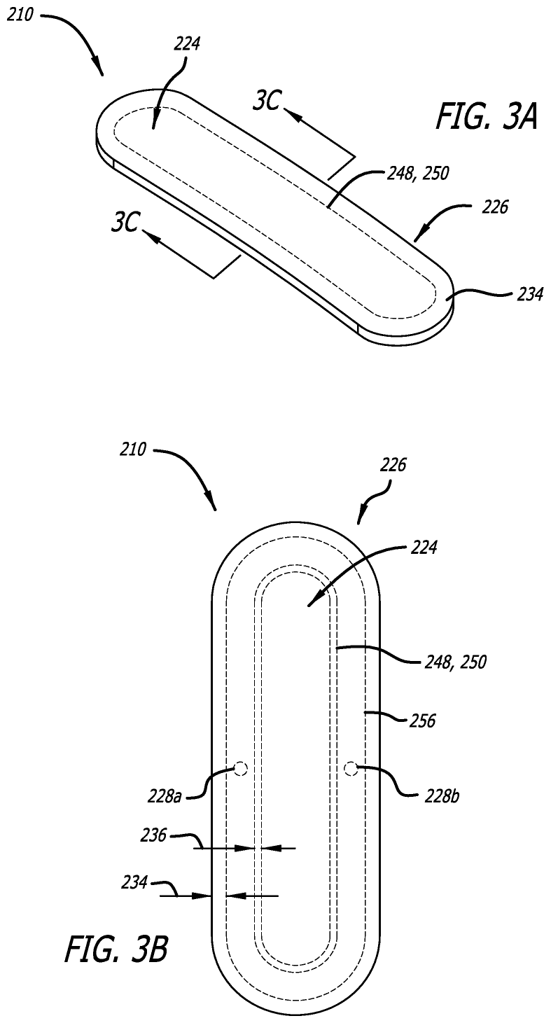

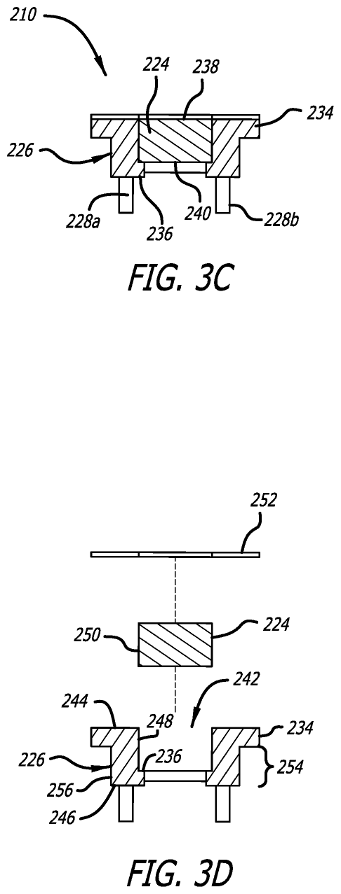

Continuing with reference to FIGS. 2A-2D, and with additional reference to FIGS. 3A-3D, the enclosure 202 with the slot 206 forms a slot antenna 208. A dielectric element 210 hermetically seals the slot 606 to provide a barrier between the interior of the neurostimulator 102 and biological fluid and tissue of the body in which the neurostimulator is implanted, and extends at least partially through the thickness of the slot 206 to provide mechanical stability to the enclosure 202 in the area of the slot. The dielectric element 210 includes a dielectric filler 224, a metallic boundary 226 that holds and surrounds the dielectric filler, and a pair of connection pins 228a, 228b that extend from the metallic boundary. The dielectric element 210 can be manufactured as a separate component that is configured to be inserted into the slot 206 of the outer wall 204 of the enclosure 202 during assembly of the neurostimulator 102.

With reference to FIGS. 3A-3D, the metallic boundary 226 has a filler opening 242 that extends through the top 244 of the metallic boundary and the bottom 246 of the metallic boundary. The filler opening 242 is shaped to receive the dielectric filler 224. To this end, the shape and size of the filler opening 242 defined by the perimeter wall 248 of the filler opening matches the shape and size of the dielectric filler 224 defined by the perimeter wall 250 of the dielectric filler such that the dielectric filler fits within the filler opening. The dielectric filler 224 can be held in place within the filler opening 242 between a lower flange 236 of the metallic boundary 226 and a top layer 252 of sealing material. Alternatively, the dielectric filler 224 can be secured in place within the filler opening 242 by brazing the dielectric filler to the metallic boundary 226, in which case the top layer 252 is an optional feature. In some instances, where the top surface 238 of the dielectric filler 224 does not align with the top 244 of the metallic boundary 226, a layer of sealing material may be applied to the top surface of the dielectric filler to provide a flush top surface to the dielectric element 210. In any case, the coupling of the dielectric filler 224 and the metallic boundary 226 makes a hermetic seal.

The metallic boundary 226 has a slot portion 254 that is shaped and sized to be received by the slot 206 of the enclosure. To this end, the shape and size of the slot portion 254 defined by the perimeter wall 256 of the slot portion matches the shape and size of the slot 206 of the enclosure 202 such that the slot portion fits within the slot. The slot portion 254 is held in place within the slot 206 of the enclosure by an upper flange 234 of the metallic boundary 226. With additional reference to FIG. 2C, held in place as such, the slot portion 254 of the metallic boundary 226 achieves mechanical alignment of the dielectric filler 224 and the slot 206 by positioning the dielectric filler in the slot 206 such that the top surface 238 of the dielectric filler is above the exterior surface 230 of the enclosure and the bottom surface 240 of the dielectric filler is below the interior surface 232 of the enclosure.

As shown in FIGS. 2C and 2D, the lower surface 235 of the upper flange 234 of the metallic boundary 226 is shaped (e.g., curved, contoured, etc.) to match the shape of the exterior surface 230 of the enclosure 202 in the area of the slot 206. As such and as shown in FIG. 2C, the respective complimentary surfaces 230 and 235 enable a flush mechanical and electrical coupling between the dielectric element 210 and the enclosure 202 that provides a hermitic seal between the components. The hermetic coupling between the dielectric element 210 and the enclosure 202 may be made by soldering, welding, or gluing with a conductive glue. In other embodiments, where the respective surfaces 230 and 235 may not match or complement each other, a hermetic seal may be provided by filling in any space or gaps between the respective surfaces with a soldering, welding, or gluing material, to provide a hermetic seal.

The metallic boundary 226 can be formed of electrically conductive material, e.g., titanium or stainless steel alloy, and is electrically coupled with the exterior surface 230 of the outer wall 204. For example, the metallic boundary 226 may be electrically coupled with the exterior surface 230 by soldering, welding, or gluing with a conductive glue. Thus, the metallic boundary 226 and the enclosure 202 form an electrically connected unit. As previously mentioned, the metallic boundary 226 provides mechanical stability to the enclosure 202 in the area around the slot 206. The metallic boundary 226, however, does not affect the operation of the slot antenna 208 in a significant way as compared to the influence of the slot 206 filled with the dielectric filler 224 and the surrounding tissue.

With reference to FIGS. 2C and 2D, the connection pins 228a, 228b that extend from the metallic boundary 226 can be formed as part of the metallic boundary or they can be separately formed and attached to the metallic boundary. In either case, the connection pins 228a, 228b extend from opposite sides of the metallic boundary 226. More specifically, a first connection pin 228a extends from a first side of the metallic boundary 226 and a second connection pin 228b extends from a second side of the metallic boundary such that upon insertion into a slot 206 the first connection pin is at a first side 218a of the slot and the second connection pin 228b is at a second side 218b of the slot, opposite the first side 218a of the slot.

The dielectric filler 224 may be formed of a non-metallic, biocompatible material (e.g., silicone, epoxy, plastic, rubber, ceramic) preferably with negligible metallic/lossy add-on at the desired wireless radio frequency band. In some embodiments, the dielectric filler 224 is formed of a non-metallic, biocompatible material that provides a hermetic seal, e.g., ceramic. In some embodiments, the dielectric filler 224 has a thickness in the range of 20 mil to 100 mil (˜ 0.5 mm to 2.5 mm). In some embodiments, the dielectric filler 224 has a dielectric constant (relative permittivity) that is a fraction of the dielectric constant of tissue at a specific radio frequency. For example, the dielectric constant of the dielectric filler 224 at a specific radio frequency may be in the range of 2 to 20, while the dielectric constant of the scalp, which covers the implant and therefore loads the antenna, is in the range of 25 to 40 at a frequency of 2.45 GHz.

Returning to FIGS. 2A and 2B, the neurostimulator 102 also includes communication circuitry 212 within the enclosure 202, and a transmission line 214 that electrically couples the slot antenna 208 and the communication circuitry. The communication circuitry 212 may support one or both of Bluetooth communication and Wi-Fi communication. The transmission line 214 includes an enclosure end 216 and a circuitry end 220. The enclosure end 216 of the transmission line 214 is electrically coupled to the outer wall 204 of the enclosure through the connection pins 228a, 228b that extend from the metallic boundary 226. The circuitry end 220 of the transmission line 214 is electrically coupled to the communication circuitry 212. In some embodiments, additional connection pins may be included to enable an additional communication circuitry to be coupled to the slot antenna 208.

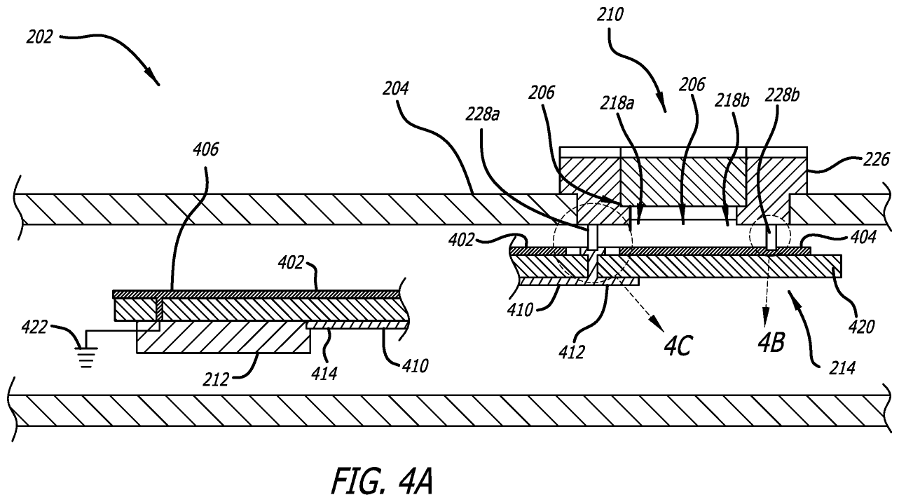

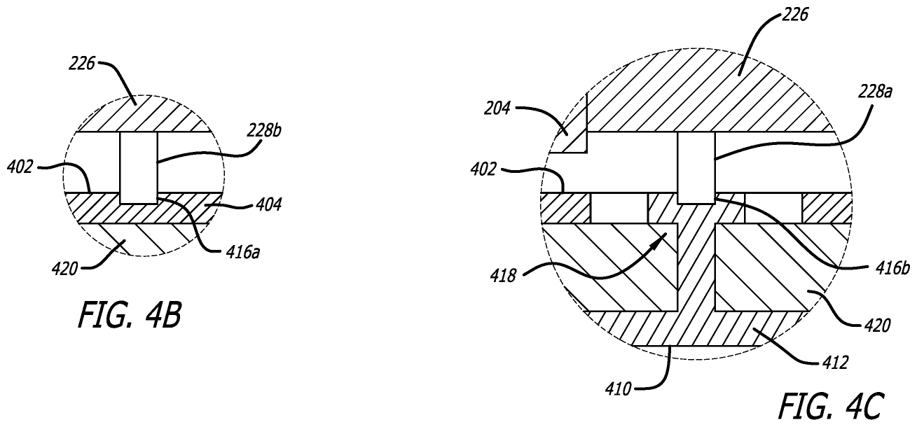

With reference to FIGS. 4A-4C, in one configuration the transmission line 214 includes a ground conductor 402 and a signal conductor 410 electrically isolated from each other by a dielectric layer 420. The dielectric layer 420 may be made of polyamide. The ground conductor 402 and the signal conductor 410 are made of copper or gold-plated copper or silver-plated copper. In this configuration of the transmission line 214, the ground conductor 402 and the signal conductor 410 are electrical traces formed on opposite surfaces of the dielectric layer 420.

The ground conductor 402 has an enclosure end 404 and a circuitry end 406. The enclosure end 404 of the ground conductor 402 is electrically coupled to the enclosure 202 at a second side 218b of the slot 206 (through an electrical connection with the second connection pin 228b of the metallic boundary 226), and the circuitry end 406 of the ground conductor 402 is electrically coupled to a ground terminal 422 of the communication circuitry 212. The signal conductor 410 has an enclosure end 412 and a circuitry end 414. The enclosure end 412 of the signal conductor 410 is electrically coupled to the enclosure 202 at a first side 218a of the slot 206 (through an electrical connection with the first connection pin 228a of the metallic boundary 226), and the circuitry end 414 of the signal conductor 410 is electrically connected or coupled to an RF signal port of the communication circuitry 212. As shown in FIG. 4A, the second side 218b of the slot 206 is opposite the first side 218a of the slot.

In other configurations the transmission line 214 may be configured such that the connections between the opposite sides 218a, 218b of the slot 206 and the communication circuitry 212 are reversed. For example, the transmission line 214 can be configured to so that the ground conductor 402 is electrically coupled to the enclosure 202 at the first side 218a of the slot 206 (through an electrical connection with the first connection pin 228a of the metallic boundary 226), and the signal conductor 410 is electrically coupled to the enclosure 202 at the second side 218b of the slot 206 (through an electrical connection with the second connection pin 228b of the metallic boundary 226).

Continuing with the transmission line 214 of FIGS. 4A-4C, regarding the ground conductor 402, in some embodiments the enclosure end 404 of the ground conductor 402 includes a contact feature 416a (shown in FIG. 4B) that is positioned to align with and electrically couple with a second connection pin 228b of the metallic boundary 226 at the second side 218b of the slot 206. The contact feature 416a can be a plated through-hole or dimple formed in the ground conductor 402. Regarding the circuitry end 406 of the ground conductor 402, with reference to FIG. 4A it is electrically connected or coupled to the ground terminal 422 of the communication circuitry 212 by a connector structure (not shown).

Regarding the signal conductor 410 and with reference to FIGS. 4A and 4C, in some embodiments the enclosure end 412 of the signal conductor 410 includes a contact feature 416b (shown in FIG. 4C) that is positioned to align with and electrically couple with the first connection pin 228a of the metallic boundary 226 at the first side 218a of the slot 206. The contact feature 416b can be a plated through-hole or dimple formed in an extension feature 418 of the signal conductor 410. The extension feature 418 can be a plated via having a horizontal leg that is co-planar with the ground conductor 402 (but electrically isolated therefrom) and a vertical leg that extends from the horizontal leg through the dielectric layer 420 and couples the horizontal leg with the portion of the signal conductor 410 beneath the dielectric layer 420. The horizontal leg includes the contact feature 416b. Regarding the circuitry end 414 of the signal conductor 410, with reference to FIG. 4A it is electrically connected or coupled to the RF signal port of the communication circuitry 212 by a connector structure (not shown).

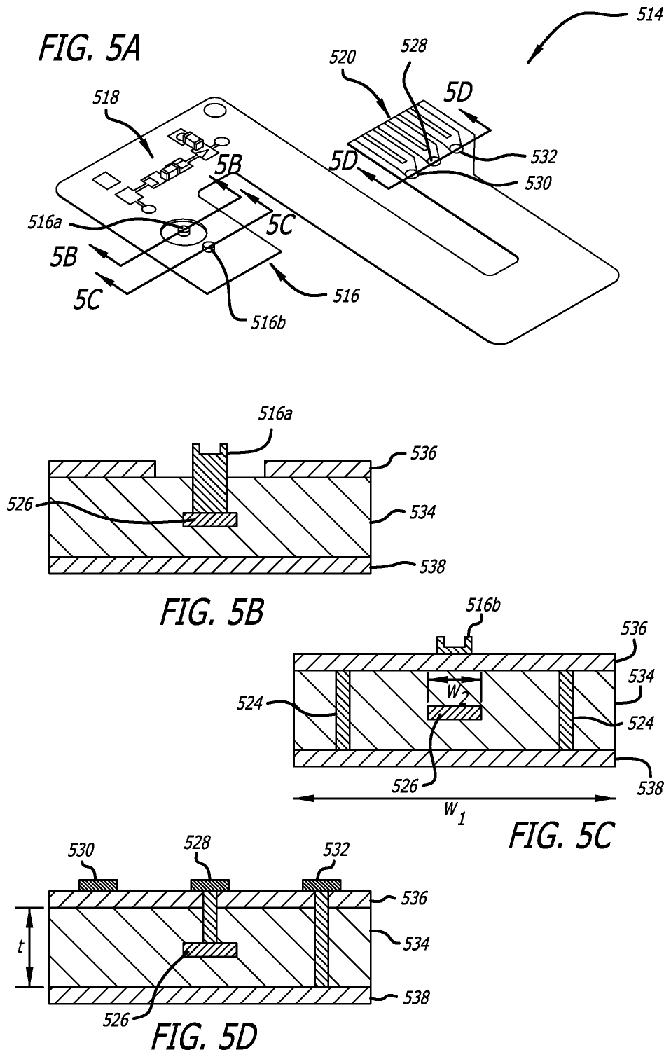

With reference to FIGS. 5A-5D, in another configuration the electrical coupling between the opposite sides 218a, 218b of the slot 206 and the communication circuitry 212 is provided by a multi-switchback shaped flexible transmission line 514. The transmission line 514 includes an enclosure connection end 516 (which generally corresponds to the enclosure end 216 of the transmission line 214 shown in FIG. 2A), a circuitry connection end 520 (which generally corresponds to the circuitry end 220 of the transmission line 214 shown in FIG. 2A), and a circuitry section 518, e.g., RF filter and/or matching network, between the connection ends. With reference to FIG. 5B, the enclosure connection end 516 includes a pair of contact features 516a, 516b.

With reference to FIG. 5B-5D, in some embodiments the transmission line 514 is a stacked arrangement of three flexible layers that include an upper layer 536 of electrically conductive material, a lower layer 538 of electrically conductive material, and an internal layer 526 of electrically conductive material. The electrically conductive material may be copper or another conductive metal or an electrically conductive alloy. As shown in FIG. 5C, electrically conductive vias 524 extend between and electrically couple the upper layer 536 and the lower layer 538 to form a two-layer ground conductor. Dielectric material 534 separates the upper layer 536, the lower layer 538, and the internal layer 526. The total thickness of the transmission line 514 is between 12-16 mils (305-405 microns).

With reference to FIG. 5B, at the enclosure connection end 516 of the transmission line 514, the contact feature 516a electrically couples with the internal layer 526. With additional reference to FIG. 2C, the contact feature 516a is arranged and configured to electrically couple with the first connection pin 228a of the metallic boundary 226. With reference to FIG. 5D, at the circuitry connection end 520 of the transmission line 514, a RF signal terminal 528 electrically couples with the internal layer 526. The RF signal terminal 528 is configured to electrically couple to an RF signal pin of the communication circuitry (not shown).

With reference to FIGS. 5C, at the enclosure connection end 516 of the transmission line 514, the contact feature 516b electrically couples with the upper layer 536 and the lower layer 538 (by the electrically conductive vias 524). With additional reference to FIG. 2C, the contact feature 516b is configured to electrically couple with the second connection pin 228b of the metallic boundary 226. With reference to FIG. 5D, at the circuitry connection end 520 of the transmission line 514, a first ground terminal 530 electrically couples with the upper layer 536 and a second ground terminal 532 electrically couples with the lower layer 538. The ground terminals 530, 532 electrically couple to a ground pin of the communication circuitry (not shown).

The two-layer ground conductor formed by the upper layer 536 and the lower layer 538, the signal conductor formed by the internal layer 526, and the dielectric material 534 are configured so the transmission line 514 is impedance matched to a signal path between the communication circuitry 212 and the slot 206 of the slot antenna 208. In one example, the transmission line 514 is matched to 50 ohms.

With reference to FIG. 5C, in one example configuration, the transmission line 514 is configured with:
an upper layer 536 and a lower layer 538, each with a cross-section width w₁ in the range of 1 mm to 10 mm,
an internal layer 526 with a cross-section width w₂ in the range of 50 microns to 500 microns,
a dielectric material 534 with a dielectric constant between 2 and 20 and a thickness t between 10 mil and 30 mil (˜ 0.25 mm to 0.76 mm).

Positioning the narrow internal layer 526 that serves as an RF signal conductor between wide upper and lower layers 536, 538 that serves as ground conductors, reduces leakage of RF signals carried by the internal layer 526.

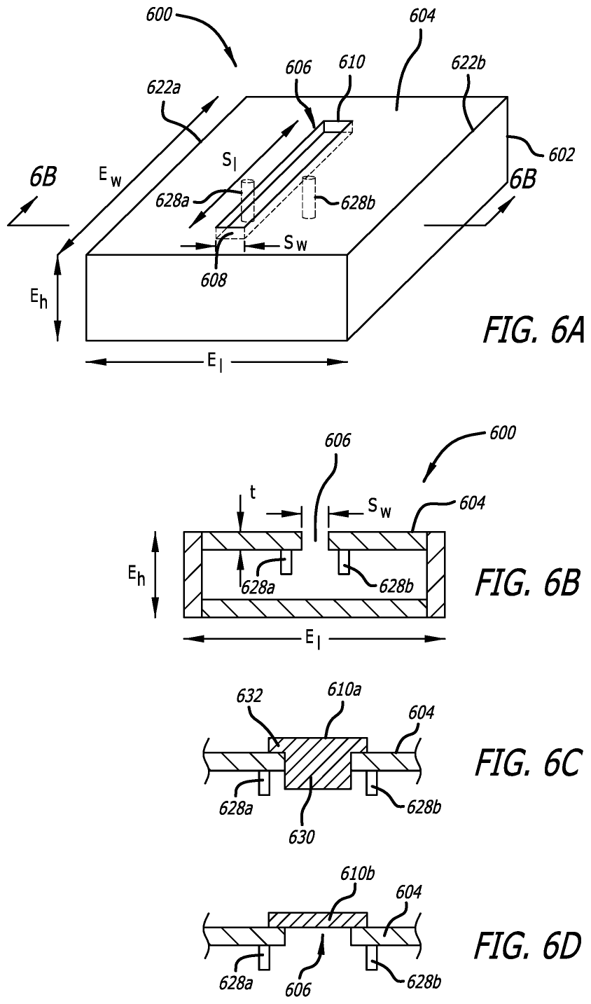

With reference to the schematic illustrations of FIGS. 6A-6D, different configurations of a slot antenna 600 that radiates/emits radio-frequency (RF) signals can be formed by a metallic enclosure 602 having an outer wall 604 and a slot 606 that extends through the outer wall. The metallic enclosure 602 may be formed of any biocompatible conductive metal, e.g., titanium, or any biocompatible conductive alloy, e.g., alloys made of titanium, stainless steel as well as other biocompatible alloy Kovar and Copper. In some embodiments the portion of the metallic enclosure 602 comprising the outer wall 604 that includes the slot 606 has a thickness t in the range of 4 mil to 30 mil (˜0.1 mm to 0.8 mm).

Conductive connection points, referred to herein as connection points 628a, 628b, are electrically coupled to the outer wall 604 of the metallic enclosure 602 and are located along the length S₁ of the slot 606. The electrical coupling between the connection points 628a, 628b and the outer wall 604 can be direct (as shown in FIG. 6B). Alternatively, as described above with reference to FIGS. 2A-2D, the electrical coupling between connection points and the outer wall can be indirect, through another component, e.g., metallic boundary 226. In either case, the connection points 628a, 628b electrically couple with communication circuitry (not shown) within the metallic enclosure 602 to feed or excite the slot antenna 600. One connection point 628a or 628b is electrically connected to an RF signal pin of the communication circuitry, while the other connection point 628b or 628a is electrically coupled to RF ground.

With reference to FIG. 6A, while the pair of connection points 628a, 628b are shown to be aligned relative to each other and near the center of the length S₁ of the slot 606, the connection points can be located anywhere along the length S₁ of the slot. Furthermore, the connection points 628a, 628b can be offset from each other along the length S₁ of the slot. As described further below, the location of the connection points 628a, 628b along the length S₁ of the slot and the location of the connection points 628a, 628b relative to each other are selected to achieve an optimum frequency response for the slot antenna 600 in the desired frequency band.

In accordance with embodiments disclosed herein, depending on the size and shape of the metallic enclosure 602, the size of the slot 606, the location of the slot within the metallic enclosure, the material (not shown) that seals the slot, and the locations of the connection points 628a, 628b along the length S₁ of the slot, the operating frequency and the resulting radiation efficiency of the slot antenna 600 can be optimized for wireless communications by communication circuitry within the metallic enclosure 602.

With continued reference to FIGS. 6A and 6B, the metallic enclosure 602 can have a length E₁ in the range of 20 mm to 75 mm and a width E_{w} in the range of 20 mm to 75 mm, and a height Eₕ in the range of 3 mm to 10 mm. The slot 606 can have a length S₁ in the range of 10 mm to 40 mm and a width S_{w} in the range of 0.5 mm to 5 mm. The interior volume of the metallic enclosure 602 is in the range of 1200 mm³ to 112,500 mm³, and the slot 606 has an area in the range of 5 mm² to 200 mm². In one configuration, the slot 606 is oriented in the outer wall 604 so that the slot length S₁ is perpendicular to the enclosure length E₁. In other configurations, the slot 606 may be placed in other, non-perpendicular orientations. In one configuration, the slot 606 is centered between the width edges 622a, 622b of the metallic enclosure 602. In other configurations, the slot 606 is off center and located closer to one of the width edges 622a, 622b of the metallic enclosure 602.

The connection points 628a, 628b are on opposite sides of the long-sides of the slot 206 in order to apply an electric field between the long sides. By placing the RF signal across the long-sides of the slot 206 an electric field is created perpendicular to the long axis of the slot antenna. The connection points 628a, 628b along the length S₁ of the slot 606 are located between a first end 608 (or first short-side) of the slot and a second end 610 (or second short-side) of the slot. The respective locations of the connection points 628a, 628b along the long-sides of the slot are part of the optimization. The different respective locations of the connection points 628a, 628b result in different impedances to excite the antenna. In some embodiments, the connection points 628a, 628b along the length S₁ of the slot 606 are located closer to the first end 608 of the slot than the second end 610 of the slot. In an example embodiment, for a slot 206 with a length S₁ of 16 mm, each of the connection points 628a, 628b is located 8 mm from the first end 608 of the slot. In some embodiments the first connection point 628a and the second connection point 628b are offset from each other along the length of the slot 606. This offset may be due to mechanical reasons or to fine tune the impedance of the slot antenna to a desired range. In an example embodiment for a slot 206 with a length S₁ of 16 mm, the first connection point 628a is located 5 mm from the first end 608 of the slot and the second connection point 628b is offset from the first connection point 628a by 7 mm.

In one design example of a slot antenna based on FIG. 6A, the enclosure 602 has a length E₁ of 30 mm, a width E_{w} of 50 mm, and a height Eₕ of 10 mm. Thus, the outer wall 604 of the enclosure 602 has an area of 1500 mm² and the interior volume of the metallic enclosure 602 is 15,000 mm³. The slot 606 has a length S₁ of 16 mm and a width S_{w} of 2 mm. Thus, the slot 606 has an area of 32 mm². The slot 606 is oriented in the outer wall 604 so that the slot length S₁ is perpendicular to the enclosure length E₁. The slot 606 is centered between the width edges 622a, 622b of the metallic enclosure 602. The first connection point 628a is located 8 mm from the first end 608 of the slot. The second connection point 628b is also located 8 mm from the first end 608 of the slot. With these design parameters the antenna performed as follows initial return loss of −3 dBi with the peak gain of around −10 dBi when implanted.

With reference to FIGS. 6C and 6D, a dielectric element 610a, 610b can be positioned relative to the slot 606 to provide a hermetic seal around the slot 606. In some embodiments, such as shown in FIG. 6C, the dielectric element 610a extends through the thickness of the slot 606. This configuration of the dielectric element 610a may be preferred because it provides mechanical support to the outer wall 604 in the area of the slot 606. In other embodiments, such as shown in FIG. 6D, the dielectric element 610b covers the slot 606 without extending into the slot. In other embodiments (not shown), the dielectric element 610a extends only partially through the thickness of the slot 606. The dielectric element 610a, 610b can be formed of a non-metallic, biocompatible material (e.g., silicone, epoxy, plastic, rubber, ceramic) with a negligible ohmic and dielectric losses at the desired wireless frequency.

In the configuration shown in FIG. 6C, the dielectric element 610a includes a lower portion 630 that is shaped and sized to fit within and extend through the slot and an upper rim 632 that extends from the lower portion to provide a surface that abuts the outer wall 604. The dielectric element 610a can be secured to the metallic enclosure 602 by brazing to provide a hermetic seal. In the configuration shown in FIG. 6D, the dielectric element 610b is shaped and sized to cover the slot 606 and includes a surface that abuts the outer wall 604. The dielectric element 610b can be secured to the metallic enclosure 602 by brazing to provide a hermetic seal.

Performance of Neurostimulator with Slot Antenna

RF performance of a slot antenna configured as disclosed herein, was modeled under simulated implant conditions. More specifically, the outer wall 204 of a neurostimulator 102 with a slot antenna 208 was simulated inside a phantom model representing a patient's head, with the slot 206 of the slot antenna 208 adjacent tissue under the scalp, and the radiation gain (i.e., the percentage/portion of the input power from the communication circuitry of the neurostimulator 102 that radiates from the slot antenna 208) was measured under these simulated conditions.

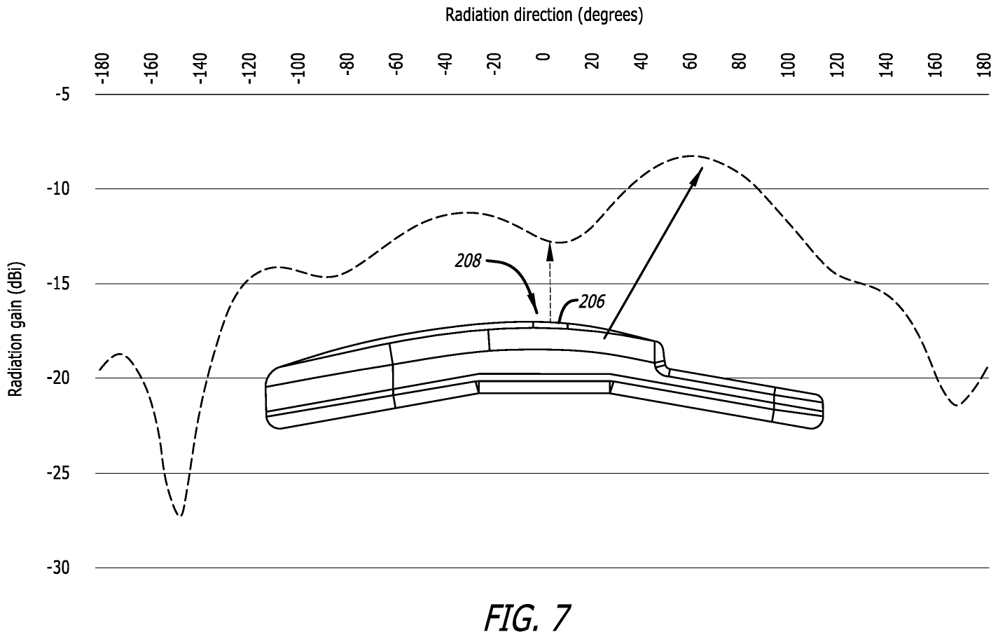

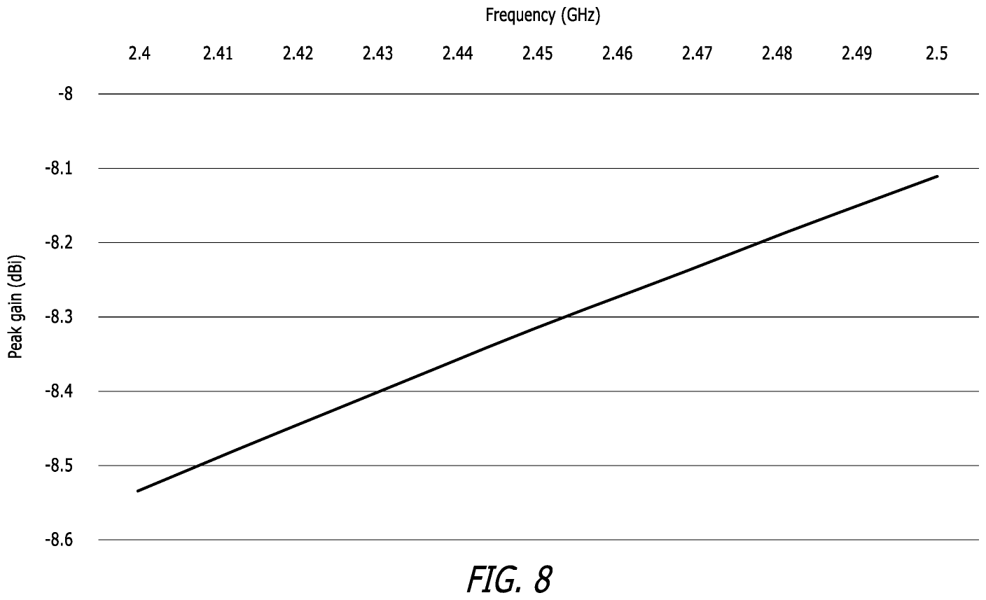

With reference to FIG. 7, radiation gain within the 2.4 GHz spectrum band (2400 to 2483.5 MHz) (e.g., Bluetooth) as function of the directional degree of RF signal radiation relative to the slot 206 of the slot antenna 208 was measured at directional degrees between −180° and +180° relative to the slot of the slot antenna, where 0° is aligned with the slot. Note, all the environmental losses are included in measures of radiation gain. Results of the simulated test show a peak radiation gain region around +60°. With additional reference to FIG. 8, results of the simulated test show that at the peak radiation gain region around +60°, the slot antenna 202 has a peak radiation gain of about −8.12 dBi (i.e., about 15%) at about 2.5 GHZ, and a slightly lower peak radiation gain of about −8.53 dBi at about 2.4 GHz.

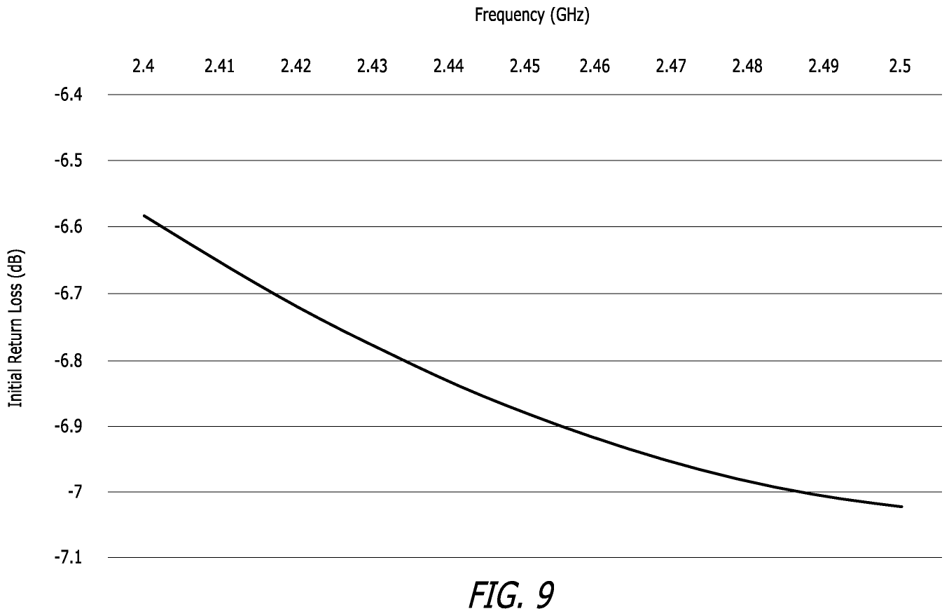

With reference to FIG. 9, initial return loss of the antenna was also measured under these simulated conditions. Return loss (i.e., matching) gives the percentage of the input power that gets transferred to the antenna. As an example, a return loss of 10 dBi is equivalent to a 10% reflection of the power back to the communication circuitry. Results of the simulated test show the slot antenna 208 has an initial return loss of ˜6.6 dBi at about 2.4 GHz, and a slightly lower initial return loss of about −7.0 at about 2.5 GHz. The return loss may be improved by the use of a matching network between the communication circuitry 212 and the slot antenna 208.

Assembly of Neurostimulator with Slot Antenna

Following is an example process of assembling a neurostimulator with a slot antenna of the type shown in FIG. 2A.

A top subassembly of the neurostimulator is assembled. To this end, a top half of an enclosure 202 is obtained. The top half of the enclosure 202 has an outer wall 204 with a slot 206 that extends through the outer wall. A dielectric element 210 is obtained and placed relative to the outer wall 204 so as to seal the slot 206. With reference to FIGS. 3A-3C, in some configurations the dielectric element 210 includes a dielectric filler 224 and a metallic boundary 226 with connection pins 228a, 228b extending from the bottom of the metallic boundary. With reference to FIGS. 2C and 2D, the dielectric element 210 is aligned with the slot 206 and the dielectric filler 224 is then placed in slot 206. The metallic boundary 226 is then coupled to the outer wall 204 of enclosure 202, thereby establishing an electrical coupling between the top half of the enclosure 202 and the metallic boundary. The coupling may be by soldering, welding, or gluing with a conductive glue. The coupling hermitically seals the slot 206.

In some embodiments, the outer wall 204 of the top half of the enclosure 202 has a cross-section profile in the area of the slot 206 and the dielectric element 210 has a cross-section profile that matches or compliments the profile of the outer wall 204 in the area of the slot. More specifically, the metallic boundary 226 of the dielectric element 210 has a profile that matches or compliments the profile of the outer wall 204 in the area of the slot 206. In some configurations, the complimentary cross-section profiles are curved, non-linear cross-section profiles. In some configurations, the complimentary cross-section profiles are flat, linear cross-section profiles. In either case, the metallic boundary 226 is in abutting contact with the outer wall 204.

In some embodiments, the outer wall 204 of the top half of the enclosure 202 has a cross-section profile in the area of the slot 206 and the dielectric element 210 has a cross-section profile different from the profile of the outer wall in the area of the slot. For example, the outer wall 204 may have a curved, non-linear cross-section profile, while the metallic boundary 226 of the dielectric element 210 has a flat, linear cross-section profile. In this case, a dielectric material, such as epoxy, may be applied to fill any spaces between the facing surfaces of the outer wall 204 and the metallic boundary 226.

A bottom subassembly of a neurostimulator is obtained. The bottom subassembly includes a bottom half of the enclosure 202 and holds various electronics, including communication circuitry 212. A transmission line 214 having an enclosure end 216 and a circuitry end 220 is electrically connected at its circuitry end 220 to the communication circuitry 212.

The top subassembly is placed over the bottom subassembly and aligned such that electrical connections are made between the connection pins 228a, 228b and the enclosure end 216 of the transmission line 214. Other connections between the top and bottom subassemblies are made and the top and bottom portions of the enclosure 202 are secured together typically by welding to form the neurostimulator.

The various aspects of this disclosure are provided to enable one of ordinary skill in the art to practice the present invention. Various modifications to exemplary embodiments presented throughout this disclosure will be readily apparent to those skilled in the art, and the concepts disclosed herein may be extended to other implantable medical devices. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. An implantable medical device (IMD) configured for implant in a body, the IMD comprising:
an enclosure formed of an electrically conductive material and comprising an outer wall and a slot extending through the outer wall;
a slot antenna comprising the enclosure;
a dielectric element that hermetically seals the slot; and
communication circuitry within the enclosure and electrically coupled with the slot antenna.

2. The IMD of claim 1, wherein the slot antenna has a peak radiation gain in a range of −8 dBi to −13 dBi when implanted under skin and operating in a frequency band of 2.4 GHz to 2.5 GHz.

3. The IMD of claim 1, wherein the slot antenna has a peak radiation gain in a range of −5 dBi to −15 dBi when implanted under skin and operating in a frequency band of 5.15 GHz to 5.85 GHz.

4. The IMD of claim 1, wherein the dielectric element comprises a dielectric material having a dielectric constant in a range of 2 to 20 in a frequency band in a range of at least one of 2.4 GHz to 2.5 GHz or from 5.15 GHz to 5.85 GHz.

5. The IMD of claim 1, wherein the dielectric element is formed of a non-metallic, biocompatible material and has a thickness in a range of a 20 mil to 100 mil (˜0.5 mm to 2.5 mm).

6. The IMD of claim 1, wherein the dielectric element is configured to extend to at least partially through a thickness of the slot.

7. The IMD of claim 1, wherein the dielectric element is configured to cover the slot without extending into the slot.

8. The IMD of claim 1, wherein the dielectric element comprises:
a dielectric filler; and
a metallic boundary that surrounds the dielectric filler.

9. The IMD of claim 8, wherein the metallic boundary is formed of electrically conductive material and is electrically coupled with the outer wall.

10. The IMD of claim 8, wherein the metallic boundary is configured to extend at least partially through a thickness of the slot.

11. The IMD of claim 10, wherein the dielectric element further comprises a plurality of connection pins that extend from the metallic boundary into the enclosure.

12. The IMD of claim 1, wherein the outer wall of the enclosure in a region of the slot is characterized by an enclosure profile and the dielectric element is characterized by a dielectric-element profile that matches the enclosure profile.

13. The IMD of claim 12, wherein the enclosure profile is non-linear with a first curvature and the dielectric-element profile is non-linear with a second curvature that matches the first curvature.

14. The IMD of claim 12, wherein the enclosure profile is flat and the dielectric-element profile is flat.

15. The IMD of claim 1, wherein the outer wall of the enclosure has a thickness in a range of 4 mils to 30 mils (˜0.1 mm up to 0.8 mm).

16. The IMD of claim 1, further comprising a transmission line electrically coupled at an enclosure end to the outer wall at each of a first connection point and a second connection point on opposite sides of the slot, and at a circuitry end to the communication circuitry.

17. The IMD of claim 16, wherein the transmission line comprises:
a signal conductor having an enclosure end electrically coupled to the first connection point, and a circuitry end electrically coupled to a signal terminal of the communication circuitry; and
a ground conductor having an enclosure end electrically coupled to the second connection point, and a circuitry end electrically coupled to a ground terminal of the communication circuitry.

18. The IMD of claim 17, wherein:
the ground conductor comprises an upper layer of electrically conductive material and a lower layer of electrically conductive material, and the upper layer is electrically coupled to the lower layer by electrically conductive vias,
the signal conductor comprises an internal layer of electrically conductive material positioned between the upper layer and the lower layer, and
the transmission line further comprises a dielectric material that separates the upper layer, the internal layer, and the lower layer.

19. The IMD of claim 18, wherein the ground conductor, the signal conductor, and the dielectric material are configured so the transmission line is impedance matched to a signal path between the communication circuitry and the slot of the slot antenna.

20. The IMD of claim 19, wherein:
the upper layer and the lower layer of the ground conductor have a cross-section width between 1 mm to 10 mm;
the internal layer has a cross-section width between 50 microns to 500 microns; and
the dielectric material has a dielectric constant between 2 and 20 and a thickness between 10 mil and 30 mil.

21. The IMD of claim 1, wherein:
the enclosure has a length E₁ in a range of 20 mm to 75 mm, a width E_{w} in a range of 20 mm to 75 mm, and a height Eₕ in a range of 3 mm to 20 mm; and
the slot has a length S₁ in a range of 10 mm to 40 mm and a width S_{w} in a range of 0.5 mm to 5 mm.

22. The IMD of claim 1, wherein:
an interior volume of the enclosure is in a range of 1200 mm³ to 112,500 mm³, and
the slot has an area in a range of 5 mm² to 200 mm².
